(19) United States
(12) Patent Application Publication
(10) Pub. No.: US 2002/0025309 A1
(43) Pub. Date: Feb. 28, 2002

(54) Stable oral pharmaceutical formulation containing an anti-infective agent and a microorganism

(75) Inventors: Rajiv Indravadan Modi, Ahmedabad (IN); Yatish Kumar Bansal, Ahmedabad (IN); Bakulesh Mafatlal Khamar, Ahmedabad (IN)

Correspondence Address: POLSTER, J. PHILIP, POLSTER, LIEDER, WOODRUFF & LUCCHESI, L.C., 763 S. NEW BALLAS RD., ST. LOUIS, MO, 63141, US

(73) Assignee: Cadila Pharmaceuticals Ltd.

(21) Appl. No.: 09/935,099

(22) Filed: Aug. 23, 2001

Related U.S. Application Data
(62) Division of application No. 09/045,890, filed on Mar. 23, 1998, now Pat. No. 6,306,391.

(30) Foreign Application Priority Data
Mar. 27, 1997 (IN) 174/BOM/97

(51) Int. Cl. A61K 45/00; A61K 31/545; A61K 31/7048; A61K 31/43; A61K 31/407; A61K 31/496

(52) U.S. Cl. 424/93.44; 514/29; 514/192; 514/200; 514/210.09; 514/253.08

(57) ABSTRACT

A stable fixed dose oral pharmaceutical formulation is provided. The formulation contains at least one anti-infective agent and at least one susceptible microorganism as active ingredients. At least one of the active ingredients is coated to provide a protective barrier around the active ingredient, the active ingredients being contained in a single pharmaceutical formulation. The formulation may be a tablet, a capsule, or a powder which may be made into a stable liquid. The protective barrier protectis the susceptible micro-organism from the effect of the anti-infective agent to maintain the susceptible micro-organism in a viable form for a period of at least three months. The anti-infective agent can be an antibiotic such as an amoxycillin and the microorganism can be Lactobacillus acidophilus.

### CROSS-REFERENCE TO RELATED APPLICATIONS

This is a division of copending application Ser. No. 09/045,890 filed Mar. 23, 1998. Priority of Indian application 174/BOM/97 filed Mar. 27,1997 is claimed under 35 U.S.C. § 119.

### STATEMENT REGARDING FEDERALLY SPONSORED RESEARCH OR DEVELOPMENT

Not Applicable.

### BACKGROUND OF THE INVENTION

The present invention relates to a process of manufacturing a formulation containing anti-infective agent(s) with viable organisms, which are susceptible to anti-infective agents. Microorganisms are used to prevent adverse effects like diarrhea caused by anti-infective agents.

The present invention is directed to a formulation wherein anti-infective agents and susceptible viable organisms are combined in such a way that microorganisms, though susceptible to anti-infective agent, remain viable for the shelf life of a formulation and/or until they are consumed. Susceptible organisms are usually combined with anti-infective agents to prevent or minimize adverse effects of anti-infective agents like diarrhea, pseudomembranous colitis, mega colon, etc.

Organisms are classified as pathogens and commensals. Pathogens are responsible for various infectious diseases and are not normally present in that part of the body. They are also known as infectious agents. Commensals are normally present in various parts of body and perform useful functions. They provide vitamin K. B-12, Thiamine. Riboflavin etc. to the body. They inhibit the growth of pathogens by a variety of mechanisms.² Anti-infective agents are used to treat or prevent infectious diseases. They kill organisms by various ways. However they are not always specific for pathogens and also kill commensals.² Destruction or reduction in the number of commensals results in loss of function of commensals and various effects of these are seen.^{2.5} These effects are known as adverse effects or side effects of anti-infective therapy. Diarrhea with or without super-infection is one of such effects seen with anti-infective therapy. 3 4 6 Diarrhea is seen as an adverse reaction to many antibiotics, but it is most commonly seen with broad-spectrum antibiotics. The incidence of diarrhea also depends on the level of absorption from the G. I. tract. It is less frequent with those getting completely absorbed compared to incompletely absorbed. It also depends on the amount of drug used. The antibiotics causing diarrhea include clindamycin. ampicillin, amoxycillin, cephalosporins (e.g. cefuroxime axetil, cefixime, cepahlexin ceftriaxone), amoxycillin+clauvanic acid, ampicillin+salbcutam, fluoroquinolens and other combinations of broad spectrum antibiotics, e.g. amoxycillin+cloxacillin. ^{3, 5, 6, 7, 8, 9, 10, 11, 12, 13, 16, 18} Diarrhea can be benign and secondary to transient dysfunction of normal colonic flora due to anti-infective agents⁶ or super-infection by pathogens like clostridium difficile following alteration of normal flora by anti-ineffective agents.^{7, 4,19, 20} Management in such an event requires cessation of anti-infective therapy^{3, 7, 4} and use of other therapies. Other therapies which can be used include different kinds of anti-infective agents e.g. metronidazole, vancomycin,^{3, 13, 8} teicoplanin and/or use of organisms like lactobacilli, biofidobacterium, saccharomyies boulardili, streptococcus thermophilus, enterococcus facecium SF 68, L Casei GG etc.^{14, 15, 16} These can be combined with whole bowel irrigation with good results.¹⁷ The organisms used⁹ eradicate or help in eradicating pathogens by a variety of mechanisms, which include production of hydrogen peroxide or inhibition or adherence of pathogens to intestinal cells. Anti-infective agents-induced diarrhea prolongs treatment and increases the cost of therapy by increased number of ¹drugs to be used, ²days of hospitalization and consultations. Sometimes it creates a life threatening situation, e.g. pseudemembranous colitis, ^{4,13,20} toxic megacolon. The organisms named above can be used to treat diarrhea when it occurs. They can also be used to prevent diarrhea.^{14, 16, 18} Commercially available preparations include lactobacillus alone (Lactiflora, Lactobacil. Lactocap, Lactovit, Sporlac) or in combination with streptococcus (Lacticyn) or Sacchromyces (Laviest). To prevent diarrhea, organisms are given along with the anti-infective agents. This requires consumption of a minimum of two different drugs, i.e. an anti-infective agent and an organism. This decreases compliance of a patient.

Attempts have been made to put organisms and anti-infective agents into one formulation. Some of these are commercially available. Lactobacillus is a commonly used organism. Anti-infective agents used in the formulation include ampicillin, (e.g. Alcillin plus from Alpine), amoxicycillin (e.g. Alox plus from Alpine), ampicillin+cloxacillin (e.g. Amplus from Jagsonpal, Elclox plus from Elder, Penmix plus from Dee Pharma, Pen plus from Systopic, Poxin Plus from Alpine), amoxicycillin+cloxacillin (e.g. Bicidal plus from Kee Pharma, Diclox from Croford Pharma, Twinclox plus from Alpine). They all are a simple admixture of anti-infective agents and susceptible organisms. However, analysis of commercially available admixtures, as well as those prepared by us revealed that organisms incorporated into the formulation does not remain viable and did not perform any useful function for which they were to be used. Neither organisms nor their activity could be detected as early as seven days after putting lactobacilli with various antibiotics like ampicillin, amoxycillin, amoxycillin+cloxacillin etc. or in a commercially available preparation. Though 60 million spores are put into formulation, none of them could be grown or demonstrated viable on glucose yeast extract agar plate. It also failed to produce lactic acid as evaluated by consumption of NaOH.

### REFERENCES

1. Gastrointestinal tracts chapter 65 in Text Book of Medical Physiology ed. Arther C. Guyton & John E. Hall, Publishers Prism Books (Pvt.) Ltd., 9th edition 1996

2. pp. 1042 anti-microbial agents, chapter 44 in the Pharmacological Basis of Therapeutics in Goodman & Gillman

3. PP-586 antibiotic associated colitis, Chapter 14 in Current Medical Diagnosis & Treatment 36th edition.

4. A. P. Ball, Chapter 7, Toxicity in antibiotic and chemotherapy seventh edition. edit. Francis O'Gerard

5. Betalactam therapy and intestinal flora, Journal of Chemother. 1995 May; 7 suppl 1: 25-31

6. Diarrhea caused by antibiotic therapy. Rev-Prat. 1996 January 15; 46 (2): 171-6

7. Antibiotic associated diarrhea in light of personal observations. Pol-Tyg-Lek. 1995 September; 50 (36): 45-9

8. Antibiotic-induced colitis. Semin-Pediatr-Surg. 1995 November; (4 (4): 215-20

9. Clostridium difficile acquisition rate and its role in nosocomial diarrhea at a University Hospital in Turkey. Eur-J-Epidemiol. 1996 August; 12 (4): 391-4

10. Risk factors associated with Clostridium difficile diarrhea in hospitalized adult patients: a case-control study—sucralfate ingestion is not a negative risk factor. Infect-Control-Hosp-Epidemiol. 1996 April; 17 (4): 232-5

11. Clinical comparison of cefuroxime axetil and amoxycillin/clavulanate in the treatment of patients with secondary bacterial infections of acute bronchitis. Clinical Ther. 1995 September-October; 17 (5): 861-74

12. Clinical comparison of cefuroxime axetil suspension and amoxycillin/lavulanate suspension in the treatment of pediatric patients with acute otitis media with effusion. Clinical Ther. 1995 September-October: 17 (5): 838-51

13. Antibiotic-associated pseudomembranous colitis: retrospective study of 48 cases diagnosed by colonoscopy. Therapy. 1996 January-February; 51 (1): 81-6

14. Biotherapeutic agents. A neglected modality for the treatment and prevention of selected intestinal and vaginal infections. JAMA 1996 March20; 275 (11): 870-6

15. The pharmacologic principles of medical practice, Krantz & Carr

16. Prevention of beta-lactam-associated diarrhea by saccharomyces boulardii compared with placibo. Am. J. Gastroenterol. 1995 March; 90 (3): 439-48

17. Whole-bowel irrigation as an adjunct to the treatment of chronic, relapsing Clostridium difficile colitis. J-Clin-Gastroenterol. 1996 April; 22 (3): 186-9

18. Prophylaxis against ampicillin-associated diarrhea with a lactobacillus preparation. Am. J. Hosp. Pharm. 1979 June; 36: 754-757

19. Clostridium difficile in antibiotic associated pediatric diarrhea. Indian Pediatr. 1994 February; 31 (2): 121-6

20. Side effects and consequences of frequently used antibiotics in clinical practice. Schweiz-Med-Wochenschr. 1996 March 30; 126 (13): 528-34

### BRIEF SUMMARY OF THE INVENTION

An object of the present invention is to combine susceptible organisms into a pharmaceutical composition containing anti-infective agents and keep them viable for the shelf life of the formulation or until it is consumed.

A further object of the present invention is to minimize side effects of anti-infective agents resulting from destruction/alteration of normal flora by providing viable organisms along with anti-infective agent(s).

A further object of the present invention is to provide a pharmaceutical composition which is effective after a longer period of storage.

A further object of the present invention is to increase compliance by reduction or elimination in side effects of anti-infective agents.

A further object of the present invention is to improve compliance by providing two drugs in one pharmaceutical composition.

A further object of the present invention is to provide an organism at a desired site.

The following specification particularly describes and ascertains the nature of this invention and manner in which it is to be performed.

The susceptible organisms are combined into the formulation in such a way that the organisms remain viable for the shelf life of a formulation in spite of being in contact with the anti-infective agent. To protect susceptible organisms from the effect of anti-infective agents, a protective barrier is created around the organisms or anti-infective agents, in such a way that the anti-infective agents cannot have an effect on the organisms. This results in viable organisms in the presence of anti-infective agents. The organism remains viable as long as the barrier is maintained. This is like applying paint or a film on a substance to prevent corrosion by isolating it from surroundings.

The present invention provides an appropriate barrier by way of a selected coating to one of the active ingredients in such a way that microorganisms are not affected by anti-infective agents. This results in a stable composition. By using an appropriate coating technique, the composition is made to remain stable over a period of 3-36 months at ambient/room temperature. The ratio of a microorganism to anti-infective agents in a composition can be 1:2 to 1:25 by weight. The ratio of 1:5 by weight is found to be optimal for the purpose. The amount of coating is dependent on the type of coating technique, dosage form i.e. capsule, tablet or liquid and desired shelf life. The microorganisms of the composition were found to be active after a variable time period. They also provided a therapeutic effect and eliminated gastro intestinal disturbances associated with anti-infective agents when evaluated in humans.

The protective barrier is selected depending on the route of administration and the dosage form of the pharmaceutical composition (anti-infective agent+organism).

The pharmaceutical composition so manufactured is evaluated for stability and efficacy.

The pharmaceutical composition so manufactured is evaluated at different test conditions of temperature and humidity (45° C., 37° C. at 80% relative humidity and ambient temperature) for time interval extending up to 12 months.

The samples of formulation were taken for study at 3-week intervals. Samples were analyzed for presence of organisms by quantitative and qualitative microbiological techniques. These values were found to be comparable with the amount of organisms introduced into the formulation.

The samples of formulation were also analyzed for presence of anti-infective agent by quantitative estimation. The values of anti-infective agents forms were found to be comparable to those introduced into the formulation.

Thus, findings indicate the presence of organisms and anti-infective agents in the same amount when the formulation was evaluated at different time interval after it was exposed to different environments.

The formulations so created were found to have improved therapeutic efficacy in term of reduction/elimination of antibiotic induced diarrhea.

### DETAILED DESCRIPTION OF THE INVENTION

Usually ampicillin causes maximum diarrhea amongst penicillin. The reported incidence is as high as 20% with ampicillins. In 40 patients when ampicillin+lactobacilli were given in a pharmaceutical composition prepared as described in this application, none of them developed diarrhea and everybody could complete the full course of antibiotic therapy. The non-development of diarrhea suggests efficacy of a new pharmaceutical composition prepared according to present invention.

### DESCRIPTION OF THE PREFERRED EMBODIMENTS

1. Following are examples of formulations containing various anti-infective agents and susceptible organisms. However, it is not intended that the scope of this invention be limited by these examples.

| | |
|---|---|
| Example I | Example II |
| Ampicillin 250 mgm | Ampicillin 500 mgm |
| Lactobacillus 60 million | Lactobacillus 60 million |
| Example III | Example IV |
| Amoxycillin 250 mgm | Amoxycillin 500 mgm |
| Lactobacillus 60 million | Lactobacillus 60 million |
| Example V | Example VI |
| Cloxacillin 250 mgm | Cloxacillin 500 mgm |
| Lactobacillus 60 million | Lactobacillus 60 million |
| Example VII | Example VIII |
| Ampicillin 250 mgm | Ampicillin 125 mgm |
| Cloxacillin 250 mgm | Cloxacillin 125 mgm |
| Lactobacillus 60 million | Lactobacillus 30 million |
| Example IX | Example X |
| Amoxycillin 250 mgm | Amoxycillin 125 mgm |
| Cloxacillin 250 mgm | Cloxacillin 125 mgm |
| Lactobacillus 60 million | Lactobacillus 30 million |
| Example XI | Example XII |
| Ampicillin 1000 mgm | Ampicillin 250 mgm |
| Sultamicin 500 mgm | Probenecid 250 mgm |
| Lactobacillus 60 million | Lactobacillus 60 million |
| Example XIII | Example XIV |
| Amoxycillin 250 mgm | Amoxycillin 500 mgm |
| Clavulanic acid 125 mgm | Probenecid 500 mgm |
| Lactobacillus 60 million | Lactobacillus 60 million |
| Example XV | Example XVI |
| Amoxycillin 250 mgm | Amoxycillin 250 mgm |
| Bromhexine 8 mgm | Carbocisteine 150 mgm |
| Lactobacillus 60 million | Lactobacillus 60 million |
| Example XVII | Example XVIII |
| Amoxycillin 500 mgm | Amoxycillin 500 mgm |
| Bromhexine 8 mgm | Carbocisteine 150 mgm |
| Lactobacillus 60 million | Lactobacillus 60 million |
| Example XIX | Example XX |
| Cephalexin 250 mgm | Cephalexin 500 mgm |
| Lactobacillus 60 million | Lactobacillus 60 million |
| Example XXI | Example XXII |
| Cephalexin 250 mgm | Cephalexin 250 mgm |
| Bromhexine 4 mgm | Probenecid 250 mgm |
| Lactobacillus 60 million | Lactobacillus 60 million |
| Example XXIII | Example XXIV |
| Cephalexin 500 mgm | Cefuroxime Axetil 125 mgm |
| Probenecid 500 mgm | Lactobacillus 60 million |
| Lactobacillus 60 million | |
| Example XXV | Example XXVI |
| Cefuroxime Axetil 250 mgm | Cefuroxime Axetil 500 mgm |
| Lactobacillus 60 million | Lactobacillus 60 million |
| Example XXVII | Example XXVIII |
| Cefixime 200 mgm | Cefixime 400 mgm |
| Lactobacillus 60 million | Lactobacillus 60 million |

In above examples anti-infective agents can be used for any therapeutic purpose, which in a therapeutic dosage causes significant adverse effects, which can be prevented by using an organism. The organism can be any which prevents or minimizes adverse reactions of anti-infective agents when taken at the same time. For prevention of diarrhea, pseudomembranous colitis it can be biofidobacterium, sacchormyces streptococcus thermophilus, enterococcus etc. instead of lactobacillus in above examples in their appropriate dosages.

2. Following are examples of providing barrier to organisms for different dosage forms. However, it is not intended that the scope of this invention be limited by these examples.

### Example I

Capsules:

i) Organisms can be lumped together and formulated into a tablet. The tablet is coated with a barrier film. The film-protected organisms are introduced into the capsule independently. An anti-infective agent is put in the capsule containing organisms protected by a barrier film. It can be vice versa.

ii) Organisms can be granulated. Granules containing organisms are coated with a barrier film. Barrier film coated granules are mixed with anti-infective agent before filling them into capsules.

### Example II

Tablets:

i) Layered Tablets:

Organisms are coated and compressed into layers of a tablet. The other layers of a tablet contains an anti-infective agent.

ii) Tablet Containing Mixture:

Granules of organisms are coated with barrier film and mixed with granulated material of anti-infective agents and compressed into a tablet.

iii) Coated Tablets:

Anti-infective agents are formulated into compressed tablets. They are coated. During coating stage organisms are introduced into the coating. The coating should be capable of protecting organisms from anti-infective agents. It can be vice versa i.e. anti-infective agent is included in coating.

iv) Composite Tablet

A tablet with a hole is produced containing anti-infective agents. The hole of the tablet is filled with organisms. The tablet so obtained may be coated for final finishing. Coating/barrier protection is not so much necessary as it is in a capsule form as long as moisture content is controlled and physical separation is maintained in a same tablet. A formulated tablet can be a dispersible tablet or a simple tablet.

### Example III

Liquid Formulations:

i) The organisms are coated with a barrier film mixed with other ingredients (dry form) of the formulation including anti-infective agents. The product is reconstituted before use by the addition of an adequate amount of liquid.

ii) The organisms are coated with barrier film and suspended in a liquid containing anti-infective agents or vice versa. The barrier film is stable in liquid formulation but disintegrates in the body due to alteration in surrounding, e.g. pH.

3. Following are examples of coating agents, which can be used in making a stable fixed dose of pharmaceutical composition containing anti-infective agent(s) and micro-organisms. However, it is not intended that the scope of this invention be limited by these examples.

| | Chemical Name | Trade Name |
|---|---|---|
| 1. | Cellulose acetate phthalate | Aquateric |
| | | CAP |
| | | Cellacefate |
| 2. | Poly (butyl methacrylate, (2-dimethyl aminoethyl) | Eudragit E 100 |
| | methacrylate, methyl methacrylate) 1:2:1 | Eudragit E 12.5 |
| 3. | Poly (ethyl acrylate, methyl methacrylate) 2:1 | Eudragit NE 30D |
| | | (formerly |
| | | Eudragit 30D) |
| 4. | Poly (methacrylic acid, methyl methacrylate) 1:1 | Eudragit L 100 |
| | | Eudragit L 12.5 |
| | | Eudragit L 12.5 P |
| 5. | Poly (methacrylic acid, ethyl acrylate) 1:1 | Eudragit L 30 |
| | | D-55 |
| | | Eudragit L |
| | | 100-55 |
| 6. | Poly (methacrylic acid, methyl methacrylate) 1; 2 | Eudragit S 100 |
| | | Eudragit S 12.5 |
| | | Eudragit S 12.5 P |
| 7. | Poly (ethyl acrylate, methyl methacrylate, | Eudragit RL 100 |
| | trimethylammonioethyl methacrylate chloride) | Eudragit RL PO |
| | 1:2:0.2 | Eudragit RL 30 D |
| | | Eudragit RL 12.5 |
| 8. | Poly (ethyl acrylate, methyl methacrylate, | Eudragit RS 100 |
| | trimethylammonioethyl methacrylate chloride) | Eudragit RS PO |
| | 1:2:0.1 | Eudragit RS 30 D |
| | | Eudragit RS 12.5 |
| 9. | Hydrogenated Castor Oil | Castrowax |
| | | Castrowax MP 70 |
| | | Castrowax MP 80 |
| | | Opalwax |
| | | Simulsol |
| 10. | Cetyl Alcohol | Crodacol C70 |
| | | Crodacol C90 |
| | | Crodacol C95 |
| 11. | Diethyl Phthalate | Kodaflex DEP |
| | | Palatinol A |
| 12. | Ethyl cellulose | Aquacoat |
| | | Ethocel |
| | | Surelease |
| 13. | Hydroxypropyl Cellulose | Klucel |
| | | Methocel |
| | | Nisso HPC |
| 14. | Hydroxypropyl Methylcellulose Phthalate | — |
| 15. | Zein | — |

4. Following are examples of methods of preparing fixed doses of stable pharmaceutical compositions. However, it is not intended that the scope of this invention be limited by these examples.

### EXAMPLE I

Double layered Tablet

A stable fixed dose combination layered tablet is prepared using the following components of which the active ingredients are anti-infective agent (s) and micro organisms. The remaining components are physiologically acceptable excipients. One of the active ingredients is coated in a coating pan by the coating process known to those skilled in the art. Excipients are also used along with one of the active ingredients (granules) during tablet making for lubrication as required for the purpose. Granules of separate active ingredients are first prepared by a process known to those skilled in the art. The separate sets of granules are then compressed on a double rotary tablet compression machine having a laying facility at a temperature below 250 C and relative humidity not more than 50% by processes known to those skilled in the art and the tablets are transferred to a coating pan for film coating to be given by using a film coating process known to those skilled in the art.

i) The relative proportion of anti-infective agents and excipients to prepare coating suspension and coating anti-infective agents before granulation:

| Ingredients | Parts by weight |
|---|---|
| Anti infective agent | 77.54% |
| Ethyl cellulose | 2.70% |
| Isopropyl alcohol | 7.42% |
| Dichloromethane | 12.34% |

ii) The relative proportion of anti-infective agents and excipients to prepare granules:

| Ingredients | Parts by weight |
|---|---|
| Anti-infective agent | 64.08% |
| Microcrystalline cellulose | 26.45% |
| Starch | 9.00% |
| Color Sunset Yellow Lake | 0.45% |
| Purified water | 0.02% |

iii) The relative proportion of excipients to be added to granules containing anti-infective agents as lubricants:

| Ingredients | Parts by weight |
|---|---|
| Sodium chloride | 31.91% |
| Polyplasdone XL | 14.89% |
| Microcrystalline cellulose | 21.28% |
| Saccharine sodium | 10.64% |
| Flavour orange | 10.64% |
| Magnesium stearate | 5.32% |
| Purified Talc | 5.32% |

iv) The relative proportion of microorganisms and excipients to prepare granules:

| Ingredients | Parts by weight |
|---|---|
| Microorganisms | 18.18% |
| Starch | 18.18% |
| Microcrystalline cellulose | 56.67% |
| Magnesium stearate | 0.91% |
| Polyplasdone XL | 3.03% |
| Sodium chloride | 3.03% |

The fixed dose layered tablet compositions, which are prepared through making use of the above described process contains the above active ingredients anti-infective agents and viable organisms in their respective therapeutic concentration. The compositions provide pharmacological effects which are complementary to the effects produced by (Prior art) each individual ingredient and are stable for a period of at least 3-36 months at ambient room temperature.

### EXAMPLE II

Capsules

Stable fixed dose combination capsules are prepared using following components of which the active ingredients are anti-infective agents and microorganisms. The remaining components are physiologically acceptable excipients. Granules of one of the active ingredients (e.g. microorganisms) are first prepared by a process known to those skilled in the art. The granules so formed are compressed into a tablet-by-tablet compression machine heaving a laying facility at a temperature below 25° C. and relative humidity not more than 50% by a process known to those skilled in the art. Tablets are transferred to a coating pan for coating to be given by a coating process known to those skilled in the art.

The remaining active ingredient is mixed with excipients and filled into gelatin capsules by a process known to those skilled in the art. Before sealing of the capsules, the coated tablet containing active ingredients is introduced into the capsules by a process known to those skilled in the art.

i) The relative proportions of anti-infective agents and excipients for filling in capsule:

| Ingredients | Parts by weight |
|---|---|
| Anti-infective agent | 91.94% |
| Pregelatinised starch | 6.24% |
| Magnesium stearate | 1.44% |
| Sodium lauryl sulfate | 0.38% |

ii) The. relative proportion of microorganisms and excipients to prepare granules is as follows:

| Ingredients | Parts by weight |
|---|---|
| Microorganism | 42.86% |
| Microcrystalline cellulose | 53.93% |
| Magnesium stearate | 1.07% |
| Colloidal silicone dioxide | 0.71% |
| Cross carmellose sodium | 1.43% |

iii) The relative proportion of excipients to prepare coating suspension for coating of a tablet containing microorganisms to be kept into a capsule:

| Ingredients | Parts by weight |
|---|---|
| Hydroxy propyl methyl cellulose pthalate | 4.37% |
| Titanium dioxide | 0.96% |
| Purified Talc | 0.19% |
| Polyethelene glycol | 0.99% |
| Isopropyl alcohol | 34.95% |
| Dichloromethane | 58.54% |

The fixed dose capsule compositions, which are prepared through making use of above described process contain the above active ingredients, anti infective agents, and viable organisms in their respective therapeutic concentrations. The compositions provide pharmacological effect, which are complementary to the effects produced by (prior art) each individual ingredient and are stable for at least 3-36 months at ambient room temperature.

### EXAMPLE III

Liquid Suspension

A stable fixed dose combination liquid tablet is prepared using the following components of which the active ingredients are anti-infective agent (s) and microorganisms. One of the active ingredients is granulated after suspending it in a coating suspension to provide granules of 100 micron or less in size by a processes known to those skilled in the art. Granules so prepared are suspended into a liquid formulation by processes known to those skilled in the art. The other active ingredient is introduced into the suspension by the process known to those skilled in the art in such a way that final concentration of microorganisms is 20% of anti-infective agent(s).

The relative proportion of anti-infective agents and excipients to prepare coated granules:

| Ingredients | Parts by weight |
|---|---|
| Anti infective agent | 56.82% |
| Cellulose acetate pthalate | 22.73% |
| Isopropyl alcohol | 6.82% |
| Dichloromethane | 13.63% |

The fixed dose liquid suspension composition, which is prepared through making use of the above described process contains the above active ingredients, anti-infective agents, and viable organisms in their respective therapeutic concentrations. The composition provides pharmacological effects, which are complementary to the effects produced by (prior art) each individual ingredient and are stable for at least 3-36 months at ambient room temperature.

### EXAMPLE IV

Dry Powder Composition to Make Liquid Composition after Reconstitution

A stable fixed dose combination of dry powder for reconstituting the liquid formulation before use is prepared using the following components of which the active ingredients are anti-infective agent(s) and micro organisms. The remaining components are physiologically acceptable excipients.

One of the active ingredients is granulated after suspending it in a coating suspension by a process known to those skilled in the art. The granules so prepared are dried and mixed with a dry powder containing another active ingredient by processes known to those skilled in the art in such a way that microorganisms are 20% of anti infective agent(s) by weight.

The relative proportion of anti infective agents and the excipients to prepare coated granules is as follows:

| Ingredients | Parts by weight |
|---|---|
| Anti infective agent(s) | 50% |
| Hydroxy propyl methyl cellulose | 45% |
| K-15 M (1,00,000 cps) | |
| Purified water | 5% |

The fixed doses of dry powder compositions, which are prepared through making use of the above described process contains the above active ingredients, anti infective agents and viable organisms in their respective therapeutic concentrations. The compositions provide pharmacological effects, which are complementary to the effects produced by (prior art) each individual ingredient and are stable for at least 3-36 months at ambient room temperature. The above composition when reconstituted by adding liquid prior to use remains stable at ambient room temperature for 3 to 7 days.

5. Following are examples of therapeutic dosages of various anti-infective agents and microorganisms. However, it is not intended that the scope of this invention be limited by these examples.

A. Anti-infective Agents

Anti infective agents can be penicillins e.g. ampicillin. amoxycillin. cloxacillin, cephalosporins e.g. cephalexin, cefadroxyl, cefuroxime axetil, cefixime, beta lactamase inhibition like clauvanic acid-macrolide like erythromycin as single ingredient or combination thereof.

i). Solid dosage forms like capsules or tablet contains anti infective agents equivalent to 125,250 or 500 mgm of the active component

ii. Liquid dosage forms usually contain anti infective agents equivalent to 125 mgm of active component in 5 ml.

B. Microorganisms, which can be used for therapeutic purposes and the dosages are as under:

| | | |
|---|---|---|
| 1. | Lactobacillus Aciophillus | 10 to 100 million |
| 2. | Lactobacillus Spores | 30-60 × 10⁶ |
| 3. | Lactobacillus Lactis | 10-500 million |
| 4. | Streptococcus thermophilus | 10 million |
| 5. | Streptococcus lactis | 10 million |
| 6. | Saccromyces cerevisea | 10 million |
| 7. | Lactobacilli GG | 10¹⁰ units |

1. A stable fixed dose oral pharmaceutical formulation comprising at least one anti-infective agent as a first active ingredient and at least one microorganism susceptible to said anti-infective agent as a second active ingredient, at least one of the first and second active ingredients being coated to provide a protective barrier around the active ingredient, the active ingredients being contained in a single pharmaceutical formulation selected from the group consisting of a tablet and a capsule, wherein said tablet or capsule contains both said anti-infective agent and said microorganism, the protective barrier protecting the susceptible micro-organism from the effect of the anti-infective agent to maintain the susceptible micro-organism in a viable form for a period of at least three months.

2. The formulation of claim 1 wherein said anti-infective agent is selected from the group consisting of Ampicillin, Amoxycillin, Cloxacillin, Clavulanic acid, Sultamicin, Cefuroxime axetil, Cefadroxyl, Cephalexin, Cefixime, Erythromycin, Ciprofloxacin, and combinations thereof.

3. The formulation of claim 1 wherein said microorganism is selected from the group consisting of Lactobacillus acidophilus, Lactobacillus spores, Lactobacillus lactis, Streptococcus thermophilus, Streptococcus lactis, Saccromyces cerevisea, Lactobacilli GG, and combinations thereof.

4. The formulation of claim 1 wherein the ratio of anti-infective agent to microorganism is in the range of 2:1 to 25:1.

5. The formulation of claim 4 wherein the ratio of anti-infective agent to microorganism is about 5:1.

6. The formulation of claim 1 wherein at least one of the anti-infective agent and the microorganism is coated with a physiologically acceptable excipient to provide granules of the anti-infective agent or the microorganism.

7. The formulation of claim 6 wherein both the anti-infective agent and the microorganisim are coated with an excipient to provide granules of the anti-infective agent and granules of the microorganism.

8. The formulation of claim 7 wherein the anti-infective agent granules and the microorganism granules are formed into a layered tablet such that one layer contains the anti-infective agent and the other layer contains the microorganism.

9. The formulation of claim 6 wherein the excipient is ethyl cellulose.

10. The formulation of claim 6 wherein the excipient is a mixture of microcrystalline cellulose and starch.

11. The formulation of claim 6 wherein the excipient is a mixture of magnesium stearate, polyplasdone XL and sodium chloride.

12. The formulation of claim 1 wherein one of the active ingredients is formed into a coated tablet, and wherein said coated tablet is contained in a capsule containing the other active ingredient.

13. The formulation of claim 12 wherein said tablet contains said microorganism admixed with physiologically acceptable excipients.

14. The formulation of claim 1 wherein the coating comprises a compound selected from the group consisting of cellulose acetate phthalate; poly(butylmethacrylate, (2-dimethyl aminoethyl) methacrylate, methyl methaacrylate); poly(ethyl acrylate, methyl methacrylate); poly(methacrylic acid, methyl methacrylate); poly(methacrylic acid, ethyl acrylate); poly(ethyl acrylate, methyl methacrylate, trimethylammonioethyl methacrylate chloride); hydrogenated Castor oil; Cetyl alcohol; diethyl phthalate; ethyl cellulose; hydroxypropyl cellulose; hydroxypropyl methylcellulose phthalate; and zein.

15. A stable fixed dose oral pharmaceutical tablet comprising at least one anti-infective agent as a first active ingredient and at least one microorganism susceptible to said anti-infective agent as a second active ingredient, each of the first and second active ingredients forming a discrete part of the tablet such that the first and second active ingredients are physically separated in the tablet and the tablet includes a first region substantially free of the second active ingredient and a second region substantially free of the first ingredient, the tablet maintaining the susceptible micro-organism in a viable form for a period of at least three months.

16. The tablet of claim 15 wherein the first region and the second region are layers of the tablet, at least one of the layers comprising a coating to provide a protective barrier around the active ingredient, the protective barrier protecting the susceptible micro-organism from the effect of the anti-infective agent.

17. The tablet of claim 15, the tablet being coated with an excipient.

18. The tablet of claim 15 wherein at least one of the first active ingredient and the second active ingredient is coated with an excipient to provide granules of at least one of the active ingredients, the granules of at least one of the active ingredients being compressed separately to form a tablet part.

19. A method of forming a liquid preparation comprising at least one anti-infective agent as a first active ingredient and at least one microorganism susceptible to said anti-infective agent as a second active ingredient, the method comprising coating at least one of the active ingredients with a coating which is sufficiently stable in the liquid formulation to maintain the susceptible micro-organism in a viable form for a period of at least three days.

20. The method of claim 19 including a step of preparing a dry formulation of the said two active ingredients wherein on of them is coated to form a barrier, and wherein the dry formulation is kept in such a way that the protective barrier protects the susceptible micro-organism from the effect of the anti-infective agent to maintain the susceptible micro-organism in a viable form for a period of at least three months in the dry formulation.

21. A process as claimed in claim 19 wherein one of the active ingredients is coated and suspended into a solution containing another active ingredient.
